(19) United States
(12) Patent Application Publication
(10) Pub. No.: US 2025/0253018 A1
(43) Pub. Date: Aug. 7, 2025

(54) TRANSFORMER-BASED MODEL FOR SEMI-STRUCTURED HIERARCHICAL DATA

(71) Applicant: Express Scripts Strategic Development, Inc., St. Louis, MO (US)

(72) Inventor: Jessie M. Allen, Durham, NC (US)

(21) Appl. No.: 18/432,611

(22) Filed: Feb. 5, 2024

(51) Int. Cl. G16H 10/60; G16H 50/20

(52) U.S. Cl. CPC G16H 10/60; G16H 50/20

(57) ABSTRACT

A method includes generating a base code embedding in a first vector space for a specified entity using at least one first machine learning model based on a set of codes corresponding to the entity from a claims datastore. The method includes generating an event embedding in a second vector space for the specified entity using a second machine learning model based on a set of historical events corresponding to the specified entity and the base code embedding, and generating a time embedding in a third vector space for the specified entity using a third machine learning model based on times between consecutive ones of the set of historical events and the base code embedding. The method includes generating an aggregated embedding for the specified entity based on the event embedding and the time embedding, and generating a prediction by supplying the aggregated embedding to a fourth machine learning model.

### FIELD

The present disclosure relates to machine learning and more particularly to a large-scale transformer-based model system that generates predictions via multiple machine learning models.

### SUMMARY

A computer-implemented method for generating a prediction for a specified entity, the method includes generating a base code embedding in a first vector space for the specified entity using at least one first machine learning model based on a set of codes corresponding to the specified entity from a claims datastore. The at least one first machine learning model is configured to generate the base code embeddings such that semantically similar codes are closer in the first vector space. The method includes generating an event embedding in a second vector space for the specified entity using a second machine learning model based on a set of historical events corresponding to the specified entity and the base code embedding. The second machine learning model is configured to generate the event embedding such that codes commonly found in a single event are closer in the second vector space. The method includes generating a time embedding in a third vector space for the specified entity using a third machine learning model based on times between consecutive ones of the set of historical events and the base code embedding. The third machine learning model is configured to generate the time embedding such that sets of similarly spaced events are closer in the third vector space. The method includes generating an aggregated embedding for the specified entity based on the event embedding and the time embedding. The method includes, in response to a query designating the specified entity, generating the prediction by supplying the aggregated embedding to a fourth machine learning model.

In other features, the set of codes includes more than 10,000 codes. The set of codes is associated with International Statistical Classification of Diseases tenth revision (ICD-10) codes. In other features, the set of codes includes more than 10,000 codes. The set of codes is associated with Current Procedural Terminology (CPT) codes.

In other features, the generating the base code embedding includes generating tokens of the set of codes, inputting the tokens into fifth machine learning model, a sixth machine learning model, and a seventh machine learning model, generating a code embedding, via the fifth machine learning model, representing a first subset of the set of codes that includes whole codes, transmitting the code embedding to an embeddings combiner, inputting the code embedding into the sixth machine learning model, generating a prefix embedding, via the sixth machine learning model, representing a second subset of the set of codes that includes partial codes, transmitting the prefix embedding to the embeddings combiner, generating a character embedding, via the seventh machine learning model, representing a third subset of the set of codes that includes character codes, transmitting the character embedding to the embeddings combiner, and generating the base code embedding, via the embeddings combiner, by concatenating the code embedding, the prefix embedding, and the character embedding.

In other features, the generating the event embedding includes aggregating portions of the base code embedding associated with visits to medical provides for a plurality of patients. In other features, the method includes, in response to the query, obtaining the aggregated embedding from a data store. In other features, the method includes transforming the prediction for display on a user device and displaying the transformed prediction on the user device.

In other features, the prediction includes a future cost prediction for the specified entity. In other features, the prediction includes a patient similarity prediction for the specified entity. In other features, the prediction includes a clinical insights prediction for the specified entity.

A computer system includes memory hardware configured to store instructions and processor hardware configured to execute the instructions. The instructions include generating a base code embedding in a first vector space for a specified entity using at least one first machine learning model based on a set of codes corresponding to the specified entity from a claims datastore. The at least one first machine learning model is configured to generate the base code embeddings such that semantically similar codes are closer in the first vector space. The instructions include generating an event embedding in a second vector space for the specified entity using a second machine learning model based on a set of historical events corresponding to the specified entity and the base code embedding. The second machine learning model is configured to generate the event embedding such that codes commonly found in a single event are closer in the second vector space. The instructions include generating a time embedding in a third vector space for the specified entity using a third machine learning model based on times between consecutive ones of the set of historical events and the base code embedding. The third machine learning model is configured to generate the time embedding such that sets of similarly spaced events are closer in the third vector space. The instructions include generating an aggregated embedding for the specified entity based on the event embedding and the time embedding. The instructions include, in response to a query designating the specified entity, generating a prediction by supplying the aggregated embedding to a fourth machine learning model.

In other features, the set of codes includes more than 10,000 codes. The set of codes is associated with at least one of International Statistical Classification of Diseases tenth revision (ICD-10) codes or Current Procedural Terminology (CPT) codes, or other medical codes greater than one thousand codes.

In other features, the generating the base code embedding includes generating tokens of the set of codes, inputting the tokens into fifth machine learning model, a sixth machine learning model, and a seventh machine learning model, generating a code embedding, via the fifth machine learning model, representing a first subset of the set of codes that includes whole codes, transmitting the code embedding to an embeddings combiner, inputting the code embedding into the sixth machine learning model, generating a prefix embedding, via the sixth machine learning model, representing a second subset of the set of codes that includes partial codes, transmitting the prefix embedding to the embeddings combiner, generating a character embedding, via the seventh machine learning model, representing a third subset of the set of codes that includes character codes, transmitting the character embedding to the embeddings combiner, and generating the base code embedding, via the embeddings combiner, by concatenating the code embedding, the prefix embedding, and the character embedding.

In other features, the generating the event embedding includes aggregating portions of the base code embedding associated with visits to medical provider for a plurality of patients. In other features, the instructions further include, in response to the query, obtaining the aggregated embedding from a data store.

In other features, the instructions further include transforming the prediction for display on a user device and displaying the transformed prediction on the user device.

In other features, the prediction includes a future cost prediction for the specified entity. In other features, the prediction includes a patient similarity prediction for the specified entity. In other features, the prediction includes a clinical insights prediction for the specified entity. In other features, the prediction includes a future medical care prediction for the specified entity.

A non-transitory computer-readable medium includes processor-executable instructions that include generating a base code embedding in a first vector space for a specified entity using at least one first machine learning model based on a set of codes corresponding to the specified entity from a claims datastore. The at least one first machine learning model is configured to generate the base code embeddings such that semantically similar codes are closer in the first vector space. The instructions include generating an event embedding in a second vector space for the specified entity using a second machine learning model based on a set of historical events corresponding to the specified entity and the base code embedding. The second machine learning model is configured to generate the event embedding such that codes commonly found in a single event are closer in the second vector space. The instructions include generating a time embedding in a third vector space for the specified entity using a third machine learning model based on times between consecutive ones of the set of historical events and the base code embedding. The third machine learning model is configured to generate the time embedding such that sets of similarly spaced events are closer in the third vector space. The instructions include generating an aggregated embedding for the specified entity based on the event embedding and the time embedding. The instructions include, in response to a query designating the specified entity, generating a prediction by supplying the aggregated embedding to a fourth machine learning model.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example transformer-based model (TBM) system.

FIGS. 5A-5D are functional block diagrams of an example embeddings generation module of the TBM system.

FIG. 5E is a functional block diagram of an example prediction generation module of the TBM system.

FIGS. 6A-6E are flowcharts of an example process for generating predictions via the TBM system.

In the drawings, reference numbers may be reused to identify similar and/or identical elements.

### DETAILED DESCRIPTION

### High-Volume Pharmacy

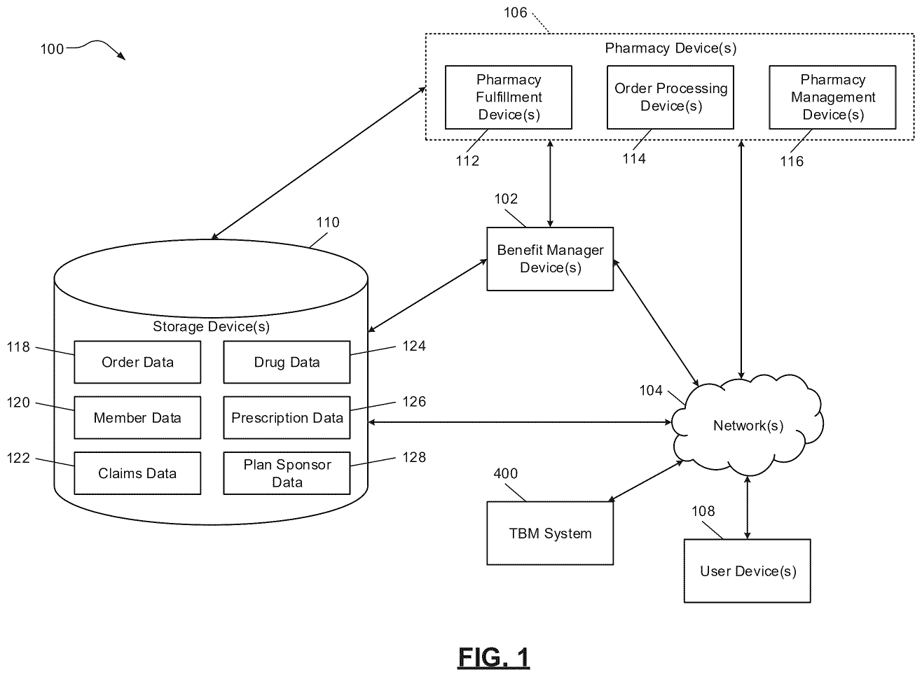

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The benefit manager device 102 may store the codes used in the present methodologies described herein.

The system 100 may also include one or more user device(s) 108. A user, such as a pharmacist, patient, data analyst, health plan administrator, medical provider, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long-term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, age, date of birth, address (including city, state, and zip code), telephone number, e-mail address, medical history, prescription drug history, etc. In various implementations, the prescription drug history may include a prior authorization claim history-including the total number of prior authorization claims, approved prior authorization claims, and denied prior authorization claims. In various implementations, the prescription drug history may include previously filled claims for the member, including a date of each filled claim, a dosage of each filled claim, the drug type for each filled claim, a prescriber associated with each filled claim, and whether the drug associated with each claim is on a formulary (e.g., a list of covered medication).

In various implementations, the medical history may include whether and/or how well each member adhered to one or more specific therapies. The medical history can include prior codes or medical treatments that can be assigned a code. The member data 120 may also include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. In various implementations, the member data 120 may include an eligibility period for each member. For example, the eligibility period may include how long each member is eligible for coverage under the sponsored plan. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122. Each of these claims may include a code to be used in the presently described methodology.

In some implementations, the claims data 122 includes claims that identify the members with whom the claims are associated. Additionally or alternatively, the claims data 122 may include claims that have been de-identified (that is, associated with a unique identifier but not with a particular, identifiable member). In various implementations, the claims data 122 may include a percentage of prior authorization cases for each prescriber that have been denied, and a percentage of prior authorization cases for each prescriber that have been approved. The prior authorization may include a code representing the medical diagnosis or the treatment for which the prior authorization is being submitted.

The drug data 124 may include drug name (e.g., technical name and/or common name), other names by which the drug is known, active ingredients, an image of the drug (such as in pill form), etc. The drug data 124 may include information associated with a single medication or multiple medications. For example, the drug data 124 may include a numerical identifier for each drug, such as the U.S. Food and Drug Administration's (FDA) National Drug Code (NDC) for each drug.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

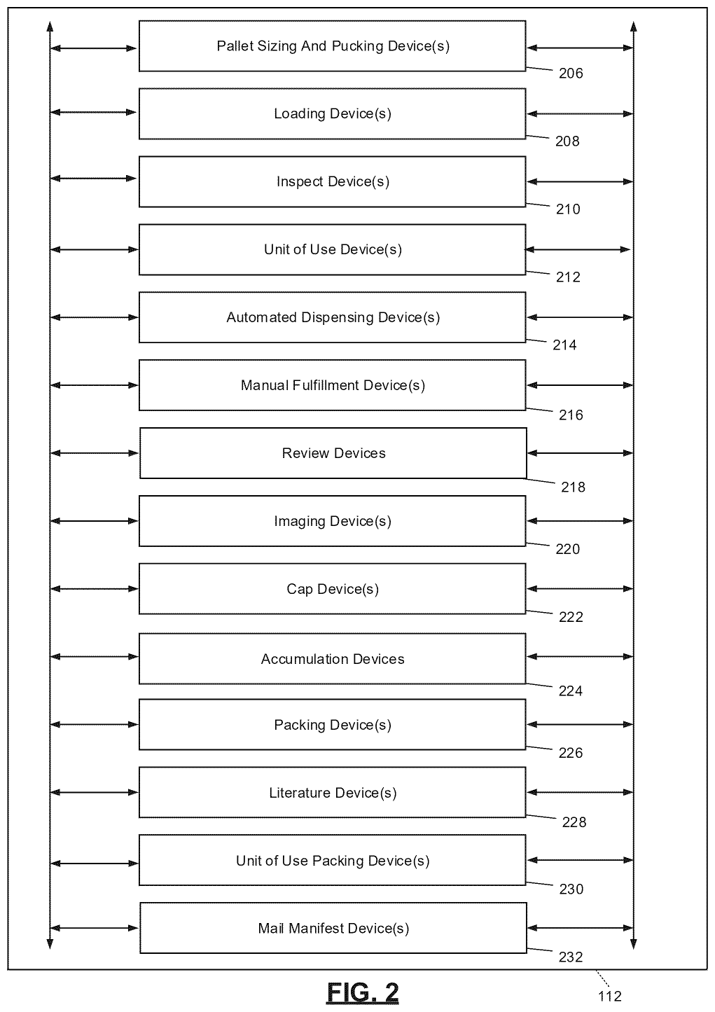

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

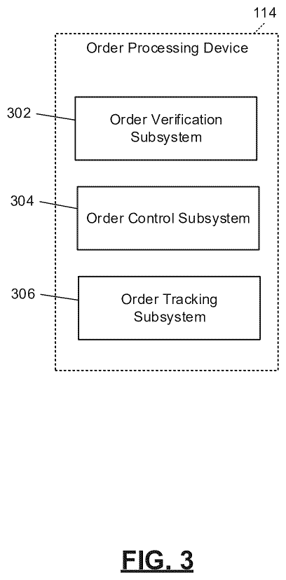

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### Transformer-Based Model System

Currently there is not a publicly available foundational machine learning model that is trained on a large repository of claims data. There is a gap in public expertise on how to effectively prepare claims data as language to be used with artificial intelligence (AI) algorithms. There is also a gap in a publicly available large repository of claims data. Accordingly, there is an opportunity to use a foundational machine learning model developed for and trained on a large volume of claims data to generate future claims related predictions including cost, utilization, disease progression, patient similarity, provider similarity, and/or clinical insights, among others.

Returning to FIG. 1, the system 100 may include a transformer-based model (TBM) system 400. The TBM system 400 is configured to retrieve, extract, and/or process a large volume of member data 120 and/or claims data 122 (for example, at least 1 GB, at least 1 TB, at least 1 PB, etc.). The TBM system 400 includes a plurality of machine learning models that use and/or are trained on at least some of the member data 120 and/or claims data 122 to generate various predictions. A prediction may include a medical efficiency prediction, a utilization prediction, a disease progression prediction, a patient similarity prediction, a provider similarity prediction, and/or a clinical insights prediction, among others. In an example, a prediction may include a cost prediction.

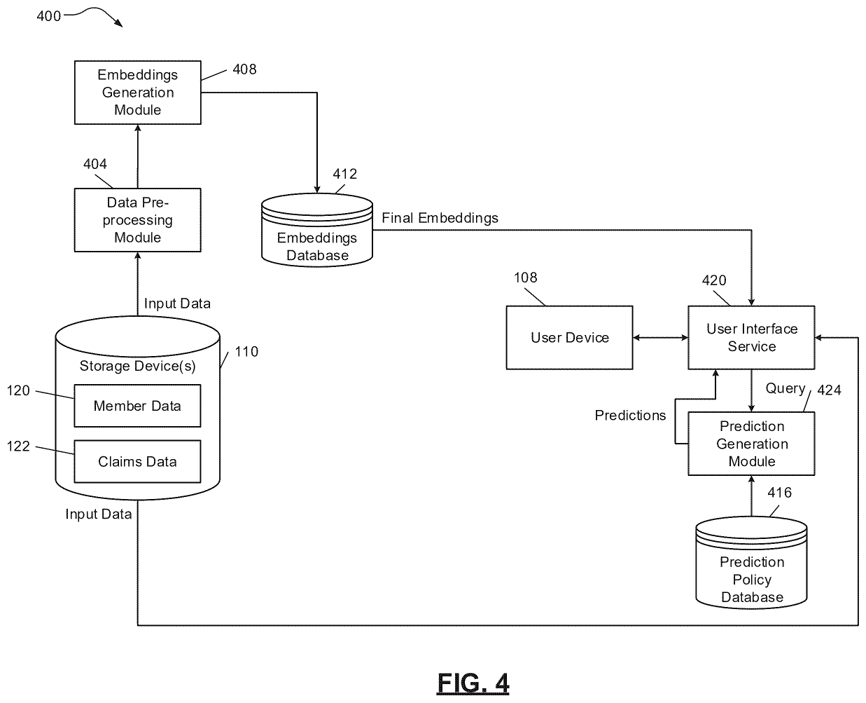

FIG. 4 is a functional block diagram of an example TBM system 400. As shown in FIG. 4, the TBM system 400 may include a data pre-processing module 404, an embeddings generation module 408, one or more databases including non-transitory computer-readable storage media, such as an embeddings database 412 and a prediction policy database 416, a user interface service 420, and a prediction generation module 424, among others.

In various implementations, the data pre-processing module 404 may be configured to receive, extract, and/or query input data (for example, member data 120, claims data 122, etc.) from one or more storage devices (for example, storage device 110). The data pre-processing module 404 may be configured to execute various processing operations on the input data from the storage device 110. In various implementations, the input data may include a large volume of the member data 120 and/or the claims data 122. The claims data 122 may include a stream of International Classification of Diseases, tenth revision (ICD-10) codes and/or Current Procedural Terminology (CPT) codes with corresponding timing data (i.e., dates data) for a plurality of individuals (i.e., patients).

In various implementations, the data pre-processing module 404 may include and/or may execute one or more base machine learning models. A base machine learning model may be configured to determine structure of the input data. For example, the base machine learning model may be configured to split and/or segment the input data to predicate whole codes, partial codes, and/or character codes in the input data. The data pre-processing module 404 may be configured to transmit at least some of the input data, output data generated via the data pre-processing module 404, and/or output data generated via the base machine learning model to the embeddings generation module 408 for further processing via the embeddings generation module 408.

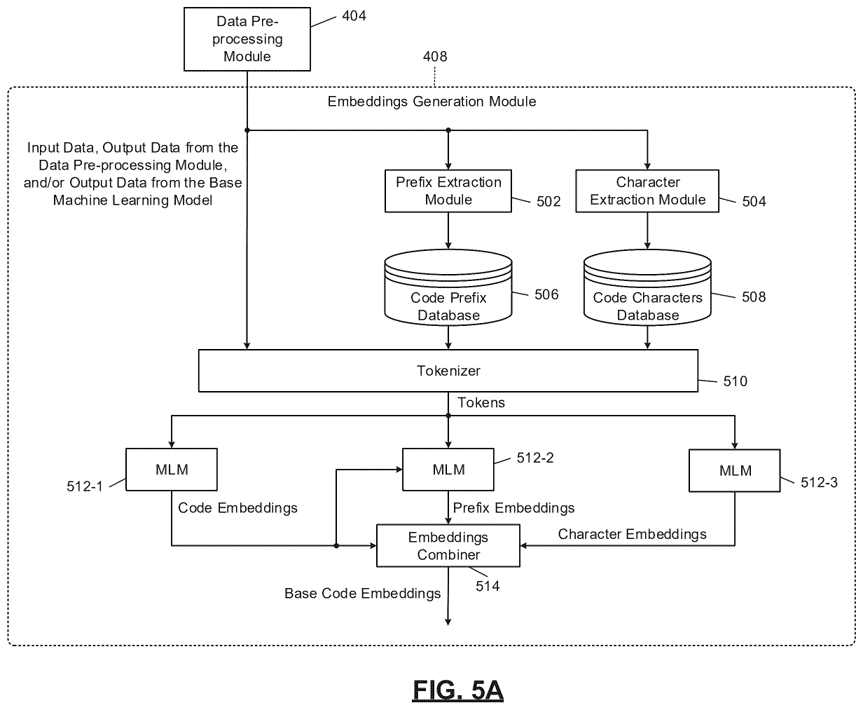

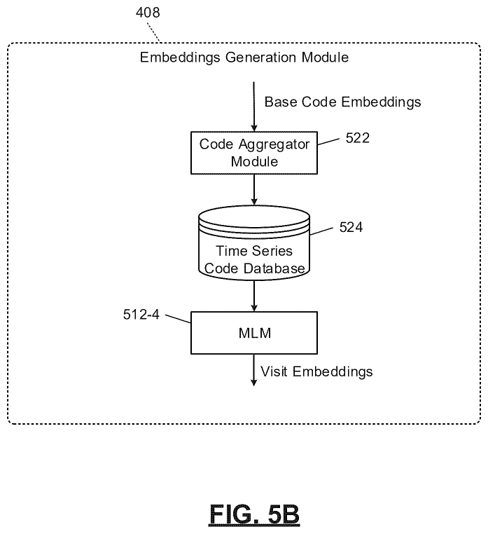

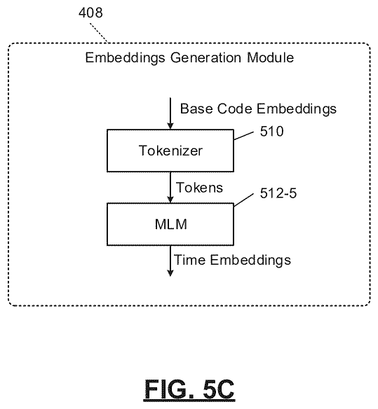

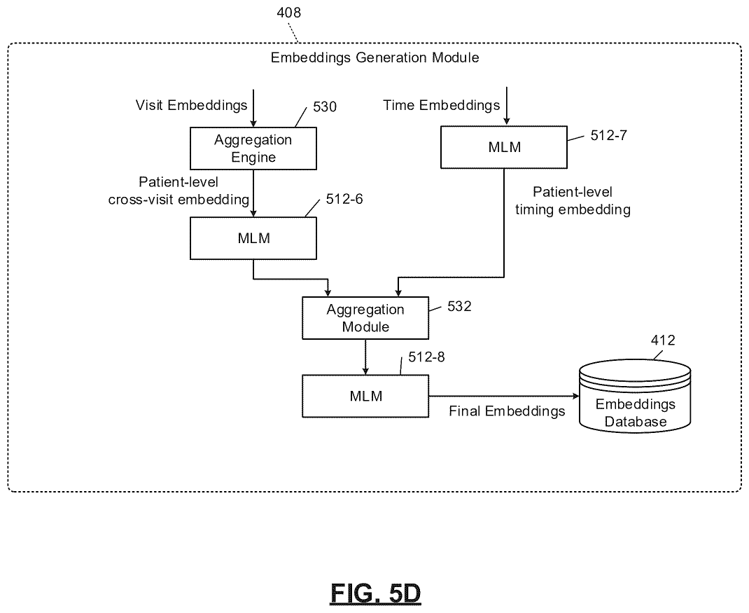

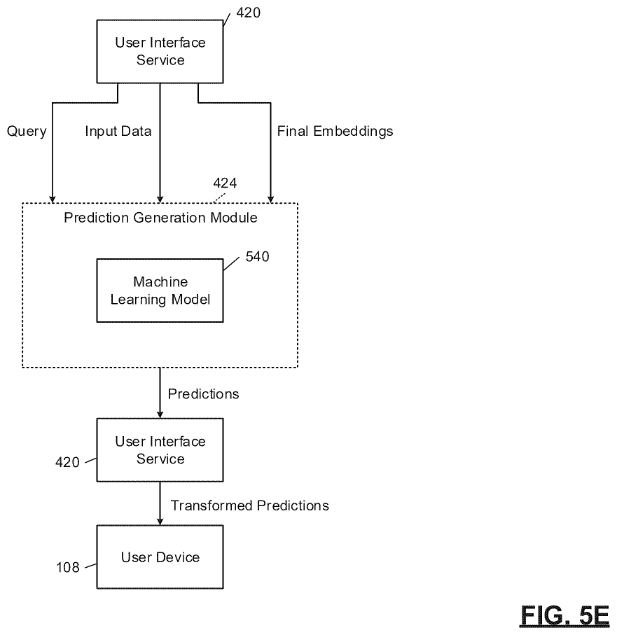

In various implementations, the embeddings generation module 408 may include and/or may execute a plurality of machine learning models (for example, Recurrent Neural Networks (RNNs), transformer-based models, and/or large language models (LLMs), among others) that are configured to generate various embeddings based on the input data. The embeddings generation module 408 may be configured to store the embeddings in the embeddings database 412 such that at least some of the embeddings may be used by other components of the TBM system 400 to execute additional operations. The plurality of machine learning models and the generated embeddings are described further below with reference to FIGS. 5A-5D.

In various implementations, the user interface service 420 may be communicatively coupled with one or more user devices (for example, user device 108). The user interface service 420 may be configured to receive inputs from the one or more user devices and may transform various outputs generated via the TBM system 400 for display on the user devices. The user interface service 420 may include a web server, one or more iOS applications, and/or one or more application programming interfaces (APIs), among others.

In various implementations, the user interface service 420 may be configured to receive queries and/or requests for predictions from a user device 108. The user interface service 420 may be configured to transform the queries and/or requests to generate corresponding prompts that may be used by and/or may automatically trigger the prediction generation module 424 and/or one or more machine learning models to execute the queries and/or requests. The user interface service 420 may be configured to receive, extract, and/or query embeddings from the embeddings database 412 to obtain the embeddings that may be used in connection with executing the queries and/or requests. The user interface service 420 may be configured to transfer the queries, the prompts, the input data, and/or the embeddings to the prediction generation module 424.

In various implementations, the prediction generation module 424 may be configured to receive the prompts, the queries, the input data, and/or the embeddings from the user interface service 420. The prediction generation module 424 may include and/or may execute one or more machine learning models to generate various predictions. The one or more machine learning models may use the prompts, the input data, and/or embeddings as inputs. In various implementations, the prediction generation module 424 may be configured to receive, extract, and query policy data from the prediction policy database 416. The policy data may include data associated with guidelines, rules, and/or principles that are created by one or more users and/or one or more organizations. The policy data may be used by the prediction generation module 424 and/or the one or more machine learning models in connection with generating the predictions. In various implementations, the prediction generation module 424 may be configured to transmit the generated predictions to the user interface service 420 for display on the user devices.

FIGS. 5A-5D are functional block diagrams of the embeddings generation module 408 that is configured to generate various embeddings. As shown in FIG. 5A, the embeddings generation module 408 may include a prefix extraction module 502, a character extraction module 504, one or more databases including non-transitory computer-readable storage media, such as a code prefix database 506 and a code characters database 508, a tokenizer 510, a plurality of machine learning models (for example, machine learning models (MLMs) 512-1, 512-2, 512-3), and/or an embeddings combiner 514, among others. In various implementations, the embeddings generation module 408 may be configured to transmit the generated embeddings to the embeddings database 412 for storage in the embeddings database 412.

With continued reference to FIG. 5A, the embeddings generation module 408 may receive at least some of the input data, the output data generated via the data pre-processing module 404, and/or the output data generated via the base machine learning model. In accordance with the base machine learning model determining and/or predicting that a first subset of the input data includes whole codes (for example, complete ICD-10 codes), the embeddings generation module 408 may be configured to transmit data associated with the first subset of input data to the tokenizer 510 for further processing via the tokenizer 510. In accordance with the base machine learning model determining and/or predicting that a second subset of the input data includes partial codes (for example, partial ICD-10 codes), the embeddings generation module 408 may be configured to transmit data associated the second subset of the input data to the prefix extraction module 502 for further processing via the prefix extraction module 502. In accordance with the base machine learning model determining and/or predicting that a third subset of the input data includes character codes (for example, ICD-10 codes that include individual letters and/or numbers), the embeddings generation module 408 may be configured to transmit data associated with the third subset of the input data to the character extraction module 504 for further processing via the character extraction module 504.

In various implementations, the prefix extraction module 502 may be configured to process and analyze the second subset of the input data to extract code prefixes from the data. In various implementations, the code prefixes may include a set of characters and/or a set of words that may be located at the beginning of variable names, function names, and/or other code identifiers. The prefix extraction module 502 may be configured to transmit the extracted code prefixes to the code prefix database 506 for storage in the code prefix database 506 and may be configured to transmit the extracted prefixes and/or at least some of the second subset of the input data to the tokenizer 510 for further processing via the tokenizer 510.

In various implementations, the character extraction module 504 may be configured to process and analyze the third subset of the input data to extract code characters from the data. In various implementations, the code characters may include a set of symbols, a set of letters, and/or a set of numbers. The character extraction module 504 may be configured to transmit the extracted code characters to the code characters database 508 for storage in the code characters database 508 and may be configured to transmit the extracted characters and/or at least some of the third subset of the input data to the tokenizer 510 for further processing via the tokenizer 510.

In various implementations, the tokenizer 510 is configured to process at least some of the input data, the extracted prefixes, and/or the extracted characters to split and/or break down the data into tokens. In various implementations, the tokens may include symbols, words, and/or characters that can be mapped (i.e., embedded) into one or more vector spaces. The tokenizer 510 may be configured to transmit the tokens to the plurality of machine learning models (for example, MLM 512-1, MLM 512-2, MLM 512-3, etc.) for further processing via the machine learning models.

In various implementations, a first machine learning model (for example, MLM 512-1) may be configured to generate code embeddings. The code embeddings may include embeddings that represent the whole codes identified in the input data. The machine learning model may be configured to transmit the code embeddings to a second machine learning model (for example, MLM 512-2) and the embeddings combiner 514. In some examples, the first machine learning model may be a self-supervising model and/or may use at least some of the tokens as an input. In some instances, the first machine learning model may be a Recurrent Neural Network (RNN).

In various implementations, the second machine learning model (for example, MLM 512-2) may be configured to generate prefix embeddings. The prefix embeddings may include embeddings that represent the code prefixes identified in the input data. The second machine learning model may be configured to transmit the prefix embeddings to the embeddings combiner 514. In some examples, the second machine learning model may be a self-supervising model and/or may use at least some of the tokens and/or the code embeddings as inputs. In some instances, the second machine learning model may be a Recurrent Neural Network (RNN).

In various implementations, a third machine learning model (for example, MLM 512-3) may be configured to generate character embeddings. The character embeddings may include embeddings that represent the code characters identified in the input data. The third machine learning model may be configured to transmit the character embeddings to the embeddings combiner 514. In some examples, the third machine learning model may be a self-supervising model and/or may use at least some of the tokens as an input. In some instances, the third machine learning model may be a Recurrent Neural Network (RNN).

In various implementations, the embeddings combiner 514 may be configured to combine the code embeddings, the prefix embeddings, and/or the character embeddings to generate base code embeddings. The base code embeddings may be associated with embeddings that represent the structure of the input data. The embeddings combiner 514 may be configured to transmit the base code embeddings to a code aggregator module 522 and the tokenizer 510 for further processing.

As shown in FIG. 5B, the embeddings generation module 408 may include the code aggregator module 522, one or more databases including non-transitory computer-readable storage media, such as a time series code database 524, and/or a fourth machine learning model (for example, MLM 512-4), among others.

In various implementations, the code aggregator module 522 may receive the base code embeddings from the embeddings combiner 514. The code aggregator module 522 may be configured to process and analyze the base code embeddings to generate time series code data.

In various implementations, the code aggregator module 522 may be configured to aggregate visit-level codes of the code embeddings to generate the time series code data. The visit-level codes may include codes associated with visits to healthcare providers for the plurality of individuals (i.e., patients). For example, an individual's visit to a healthcare provider may have one or more corresponding visit-level codes. Each visit may include a primary code including a primary diagnosis code (for example, an ICD-10 code) that represents the main reason for the visit. Each visit may include one or more secondary codes. A secondary code (for example, an ICD-10 code) may include an additional code that complements the primary code. For example, the secondary code may provide information about other conditions, complications, and/or services associated with the respective visit. In some examples, each visit may include up to 25 secondary codes.

In various implementations, the time series code data may include the visit-level codes that have been aggregated to the patent-level for each of the individuals. For example, the time series code data for each individual may represent all of the codes associated with the individual's visits to the healthcare providers. To aggregate the visit-level codes to the patient-level for each individual, the code aggregator module 522 may be configured to aggregate the individual's base code embeddings associated with the primary codes for each visit to generate an overall primary code value. The code aggregator module 522 may configured to calculate an average of all of the individual's base code embeddings associated with the primary codes and the secondary codes to generate an overall visit average value. The code aggregator module 522 may be configured to calculate a sum of all of the individual's base code embeddings associated with the primary codes and the secondary codes to generate an overall visit sum value. The code aggregator module 522 may be configured to determine a max value of all of the individual's base code embeddings associated with the primary codes and the secondary codes to generate an overall visit max value. The code aggregator module 522 may be configured to concatenate the overall primary code value, the overall visit average value, the overall visit sum value, and the overall visit max value to generate the patent-level code for the individual.

In various implementations, the code aggregator module 522 may be configured to extract the codes and/or the base embeddings associated with the codes for each visit for each individual and may transmit the extracted codes and/or embeddings associated with the codes to the time series code database 524 for storage in the time series code database 524. In various implementations, the code aggregator module 522 may be configured to determine which extracted codes are likely to be found together and/or which extracted codes are unlikely to be found together. For examples, the codes that are likely to be found together may be associated with embeddings that are similar in a dimensional embedding space and the codes that are unlikely to be found together may be associated with embedding that are dissimilar in the dimensional embedding space. In various implementations, the code aggregator module 522 may be configured to transmit the time series code data to the time series code database 524 for storage in the time series code database 524.

In various implementations, the embeddings generation module 408 may be configured to transmit the extracted codes and/or the embeddings associated with the codes, and/or the time series code data to the fourth machine learning model (for example, MLM 512-4) for further processing via the fourth machine learning model. In various implementations, the fourth machine learning model may be configured to generate visit embeddings 526. The visit embeddings 526 may include embeddings that represent the visits of the individuals. The fourth machine learning model may be configured to transmit the visit embeddings to an aggregation engine 530. The aggregation engine 530 is described further herein with reference to FIG. 5D. In some examples, the fourth machine learning model may be a self-supervising model and/or may use the extracted codes and/or the embedding associated with the code, and/or the time series code data as inputs.

With reference to FIG. 5C, in various implementations, the tokenizer 510 may receive the base code embeddings from the embeddings combiner 514. The tokenizer 510 may be configured to process the base code embeddings to split and/or break down the base code embeddings into tokens. The tokenizer 510 may be configured to transmit the tokens to a fifth machine learning model (for example, MLM 512-5) for further processing via the fifth machine learning model.

In various implementations, the fifth machine learning model may be configured to generate time embeddings. The time embeddings may include embeddings that represent times the visits occurred. In some examples, the fifth machine learning model may be a self-supervising model and/or may use the tokens generated via the tokenizer 510 as an input.

As shown in FIG. 5D, the embeddings generation module 408 may include the aggregation engine 530, a plurality of other machine learning models (for example, MLM 512-6, MLM 512-7, MLM 512-8), and/or an aggregation module 532, among others. In various implementations, the visit embeddings may be transmitted to the aggregation engine 530 for further processing via the aggregation engine 530.

In various implementations, the aggregation engine 530 may be configured to aggregate the visit embeddings for each individual and may transmit an aggregation engine output to a sixth machine learning model (for example, MLM 512-6) for further processing via the sixth machine learning model. The aggregation output may include the visit embeddings that have been aggregated to the patient-level for each of the individuals. For example, to aggregate the visit-level embeddings to the patient-level for each individual, the aggregation engine 530 may be configured to aggregate the individual's visit embeddings associated with the primary codes for each visit to generate an overall primary code value. The aggregation engine 530 may configured to calculate an average of all of the individual's visit embeddings associated with the primary codes and the secondary codes to generate an overall visit average value. The aggregation engine 530 may be configured to calculate a sum of all of the individual's visit embeddings associated with the primary codes and the secondary codes to generate an overall visit sum value. The aggregation engine 530 may be configured to determine a max value of all of the individual's visit embeddings associated with the primary codes and the secondary codes to generate an overall visit max value. The aggregation engine 530 may be configured to concatenate the overall primary code value, the overall visit average value, the overall visit sum value, and the overall visit max value to generate the patent-level embeddings for the individual.

In various implementations, the sixth machine learning model may be configured to generate a visit output (e.g., a patient-level cross-visit embedding) and may transmit the visit output to the aggregation module 532. The visit output may include an embedding representing the medical context content of a series of patent visits. In some examples, the sixth machine learning model may be a self-supervising model and/or may use the aggregation engine output as an input.

In various implementations, a seventh machine learning model (for example, MLM 512-7) may receive the time embeddings from the fifth machine learning model 512-5 for further processing via the seventh machine learning model. The seventh machine learning model may be configured to generate a time output (e.g., a patient-level timing embedding) and may transmit the time output to the aggregation module 532. The time output may include an embedding representing the time context of a series of medical visits. In some examples, the seventh machine learning model may be a self-supervising model and/or may use the time embeddings as an input.

In various implementations, the aggregation module 532 may be configured to combine the visit output and the time output to generate an aggregation module output. The aggregation module 532 may be configured to transmit the aggregation module output to an eighth machine learning model (for example, MLM 512-8) for further processing via the eighth machine learning model. The eighth machine learning model may be configured to generate final embeddings 534. The final embeddings 534 may include embeddings that represent each individual's entire medical history including all of the individual's visits and the times the visits occurred in chronological order. In some examples, the eighth machine learning model may be a self-supervising model and/or may use the visit output and the time output as inputs. The eighth machine learning model may be configured to transmit the final embeddings to the embeddings database 412 for storage in the embeddings database 412. In various implementations, the final embeddings 534 may represent a new data source that are used and/or read by other components of the TBM system 400 to generate predictions.

FIG. 5E is a functional block diagram of the prediction generation module 424 that may be configured to generate predictions based on the final embeddings. In various implementations, the prediction generation module 424 may receive the prompts, the queries, the input data, and/or the final embeddings from the user interface service 420. The prediction generation module 424 may include and/or execute a machine learning model 540. The machine learning model 540 may be configured be configured to generate various predictions and/or may use the prompts, the input data, and/or the final embeddings as inputs.

In various implementations, the machine learning model 540 may be configured to generate cost predictions, patient similarity predictions, provider similarity predictions, and/or clinical insights predictions, among others. For example, the machine learning model 540 may be configured to predict future costs for a specific individual (for example, a specific patient). In this instance, the machine learning model 540 may use final embeddings associated the specific individual's visits and times the visits occurred and/or final embeddings associated with a plurality of other individuals who have similar medical histories as the specific individual as inputs. The prediction (i.e., the machine learning model 540 output) may include the future costs (for example, dollar amount of the future costs).

In various implementations, the machine learning model 540 may be configured to predict a specific individual's future healthcare provider visits. In this example, the machine learning model 540 may use final embeddings associated the specific individual's visits and times the visits occurred and/or final embeddings associated with a plurality of other individuals who have similar medical histories as the specific individual as inputs. The prediction (i.e., the machine learning model 540 output) may include the ICD-10 codes associated with the specific individual's future healthcare provider visits. In some examples, the prediction may include a set of ICD-10 codes and the likelihood that each visit will occur.

In various implementations, the machine learning model 540 may be configured to predict future costs for a new individual (for example, an individual with no available medical data). In this additional example, the prediction generation module 424 and/or the user interface service 420 may use a lookup table associated with the embeddings database 412 and/or may query the embeddings database 412 to select final embeddings associated with a plurality of individuals who are most similar to the new individual. The prediction generation module 424 may be configured to calculate an average of the final embeddings associated with the similar individuals and may use the calculated average as an input to the machine learning model 540. The prediction (i.e., the machine learning model 540 output) may include the future costs (for example, dollar amount of the future costs).

In various implementations, the machine learning model 540 may be configured to transmit the predictions to the user interface service 420. The user interface service 420 may be configured to transform the predictions in a format suitable for display on the one or more user devices 108. The one or more user devices 108 may be configured to display the transformed predictions.

### Flowcharts

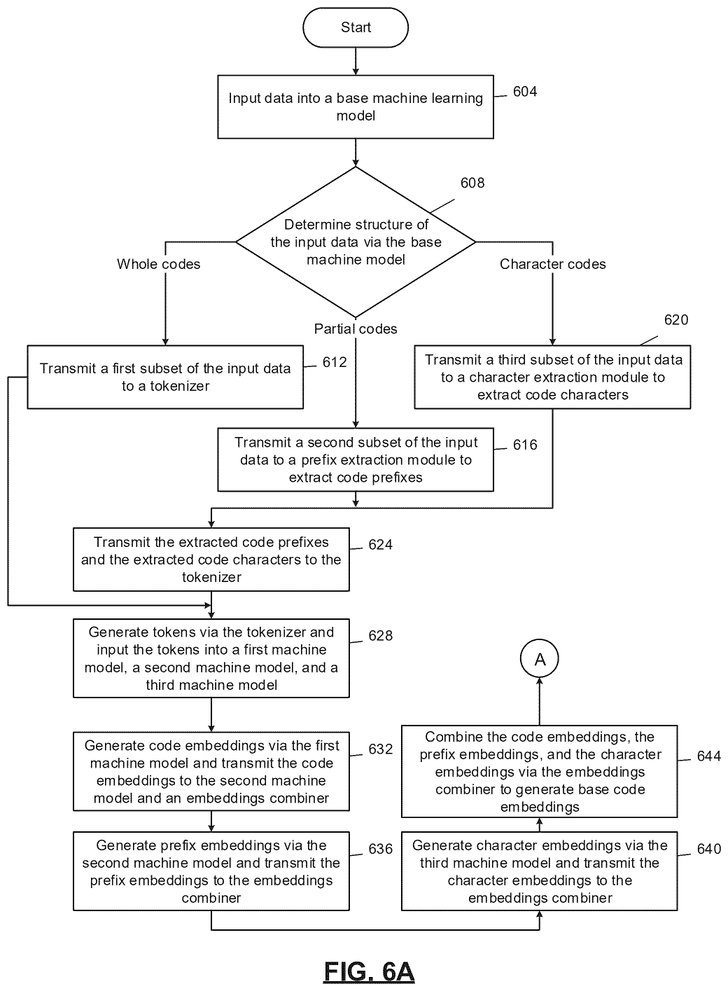

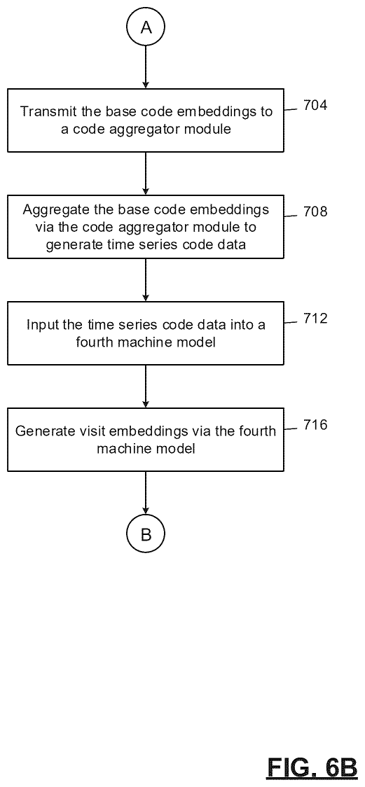

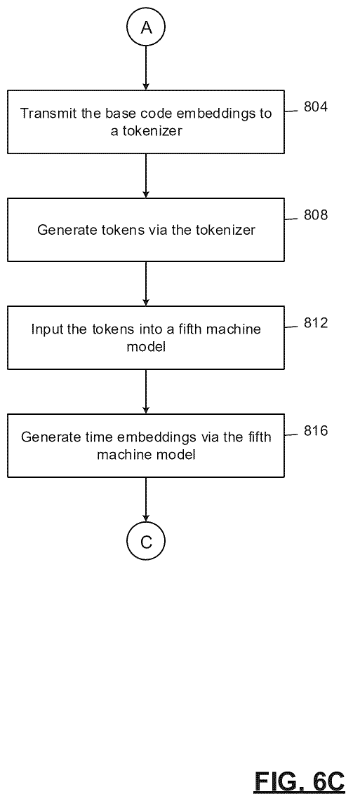

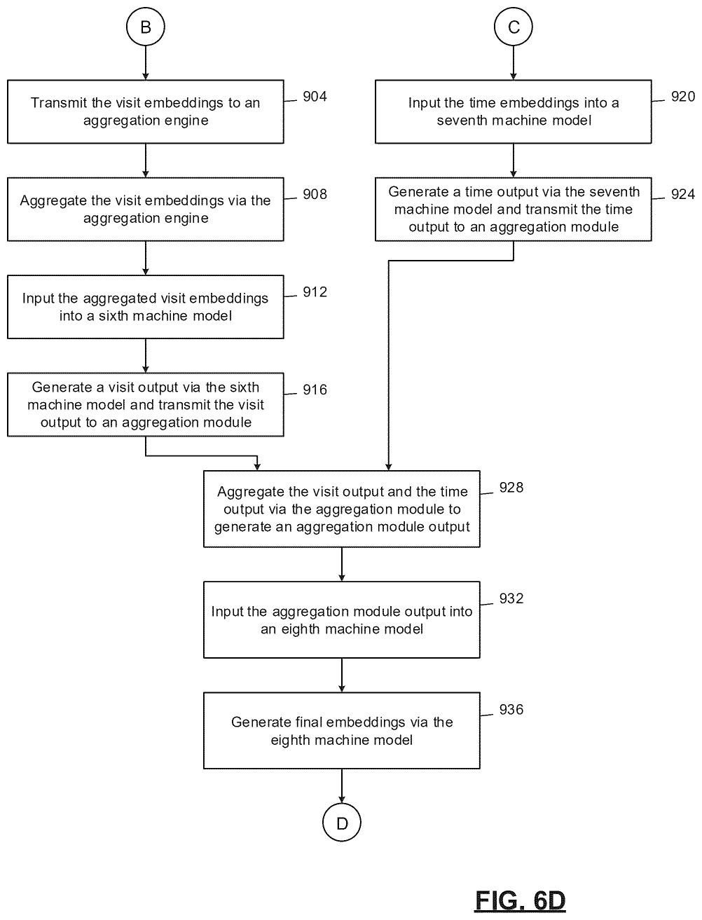

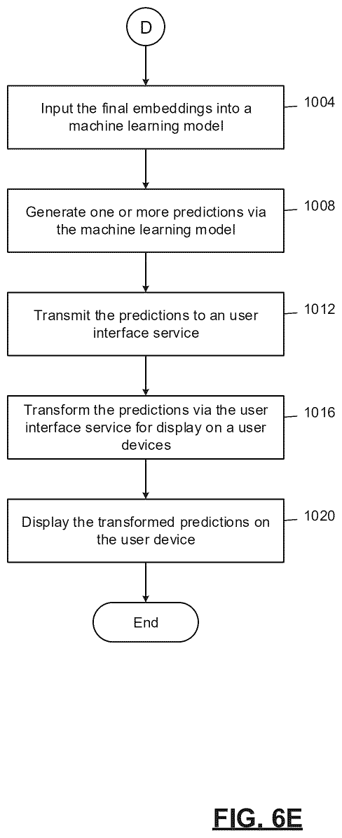

FIGS. 6A-6E are flowcharts of an example method for generating predictions via the TBM system 400. With reference to FIG. 6A, control begins at 604. At 604, input data (for example, member data 120 and/or claims data 122) may be inputted into a base machine learning model. Control proceeds to 608. At 608, the base machine learning model may determine structure of the input data. In accordance with the base machine learning model determining that a first subset of the input data include whole codes (for example, complete ICD-10 codes) at 608, control proceeds to 612. At 612, the base machine learning model may transit the first subset of the input data to a tokenizer 510.

In accordance with the base machine learning model determining that a second subset of the input data includes partial codes (for example, partial ICD-10 codes) at 608, control proceeds to 616. At 616, the base machine learning model may transmit the second subset of the input data to a prefix extraction module 502 to extract code prefixes from the second subset of input data. In accordance with the base machine learning model determining that a third subset of the input data includes character codes (for example, ICD-10 codes that include individual letters and/or numbers) at 608, control proceeds to 620. At 620, the base machine learning model may transmit the third subset of input data to a character extraction module 504 to extract code characters from the third subset of the input data. Control proceeds to 624.

At 624, the prefix extraction module 502 may transmit the extracted code prefixes to the tokenizer 510 and the character extraction module 504 may transmit the extracted code characters to the tokenizer 510. Control proceeds to 628. At 628, the tokenizer 510 may generate tokens based on the first subset of input data, the code prefixes, and the code characters. The tokenizer 510 may input the tokens into a first machine learning model 512-1, a second machine learning model 512-2, and a third machine learning model 512-3. Control proceeds to 632. At 632, the first machine learning model 512-1 may generate code embeddings and may input the code embeddings to the second machine learning model 512-2 and may transmit the code embeddings to an embeddings combiner 514. Control proceeds to 636.

At 636, the second machine learning model 512-2 may generate prefix embeddings and may transmit the prefix embeddings to the embeddings combiner 514. Control proceeds to 640. At 640, the third machine learning model 512-3 may generate time embeddings and may transmit the time embeddings to the embeddings combiner 514. Control proceeds to 644. At 644, the embeddings combiner 514 may combine the code embeddings, the prefix embeddings, and the character embeddings to generate base code embeddings. Control proceeds to 704 of FIG. 6B and 804 of FIG. 6C.

With reference to FIG. 6B, at 704 the embeddings combiner 514 may transmit the base code embeddings to a code aggregator module 522. Control proceeds to 708. At 708, the code aggregator module 522 may aggregate the based code embeddings to generate time series code data. Control proceeds to 712. At 712, the code aggregator module 522 may input the time series code data into a fourth machine learning model 512-4. Control proceeds to 716. At 716, the fourth machine learning model 512-4 may generate visit embeddings. Control proceeds to 904 of FIG. 6D.

With reference to FIG. 6C, at 804 the embeddings combiner 514 transmit the base code embeddings to the tokenizer 510. Control proceeds to 808. At 808, the tokenizer 510 may generate tokens based on the base code embeddings. Control proceeds to 812. At 812, the tokenizer 510 may input the tokens into a fifth machine learning model 512-5. Control proceeds to 816. At 816, the fifth machine learning model 512-5 may generate time embeddings. Control proceeds to 920 of FIG. 6D.

With reference to FIG. 6D, at 904 the fourth machine learning model 512-4 may transmit the visit embeddings to an aggregation engine 530. Control proceeds to 908. At 908, the aggregation engine 530 may aggregate the visit embeddings. Control proceeds to 912. At 912, the aggregation engine may input the aggregated visit embeddings into a sixth machine learning model 512-6. Control proceeds to 916. At 916, the sixth machine learning model 512-6 may generate a visit output and may transmit the visit output to an aggregation module 532. Control proceeds to 920.

At 920, the fifth machine learning model 512-5 may input the time embeddings into a seventh machine learning model 512-7. Control proceeds to 924. At 924, the seventh machine learning model 512-7 may generate a time output to the aggregation module 532. Control proceeds to 928.

At 928, the aggregation module 532 may aggregate the visit output and the time output to generate an aggregation module output. Control proceeds to 932. At 932, the aggregation module 532 may input the aggregation module output into an eighth machine learning model 512-8. Control proceeds to 936. At 936, the eighth machine learning model 512-8 may generate final embeddings. Control proceeds to 1004 of FIG. 6E.

With reference to FIG. 6E, at 1004 a user interface service 420 may input the final embeddings into a machine learning model 540. Control proceeds to 1008. At 1008, the machine learning model 540 may generate one or more predictions. Control proceeds to 1012. At 1012, the machine learning model 540 may transmit the predictions to a user interface service 420. Control proceeds to 1016. At 1016, the user interface service 420 may transform the predictions to a format that is suitable for display on a user device. Control proceeds to 1020. At 1020, the user device may display the transformed predictions. Then control ends.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Various embodiments described herein refer to International Classification of Diseases, tenth revision (ICD-10) codes. It is with the scope of some embodiments to perform the functions on prior ICD codes or other similar codes relating to medical care.

The predictive modelling described herein may use a large language model (LLM) or generative artificial intelligence for one or more of the predictive models described herein. In an example, a first LLM is applied to the selected inquiry, and one or more keywords or vectors are fed to a second LLM. The second LLM can perform one or more tasks based on the keywords or vectors to provide results or responses for presentation to a device or fed to subsequent LLMS. A respective machine learning model, such as a generative AI machine learning model, can implement each LLM.

Generative artificial intelligence (AI) is a term that may refer to any artificial intelligence that can create new content from training data. For example, generative AI can produce text, images, video, audio, code, or synthetic data that are similar to the original data but not identical. In some cases, generative AI can include or implement large language models (LLMs). The generative AI and/or LLMs receive a prompt (including instructions) and a set of data to process based on the prompt. The generative AI and/or LLMs process the data in accordance with the instructions of the prompt and generate an output that includes modifications of the set of data based on prior knowledge of the generative AI and/or LLMs.

Some of the techniques that may be used in generative AI are:
a. Convolutional Neural Networks (CNNs): CNNs are commonly used for image recognition and computer vision tasks. They are designed to extract features from images using filters or kernels that scan the input image and highlight important patterns. CNNs may be used in object detection, facial recognition, and autonomous driving applications.
b. Recurrent Neural Networks (RNNs): RNNs are designed for processing sequential data, such as speech, text, and time series data. They have feedback loops that allow them to capture temporal dependencies and remember past inputs. RNNs may be used in speech recognition, machine translation, and sentiment analysis applications.
c. Generative adversarial networks (GANs): These models consist of two neural networks: a generator and a discriminator. The generator tries to create realistic content that can fool the discriminator, while the discriminator tries to distinguish between real and fake content. The two networks compete with each other and improve over time. GANs may be used in image synthesis, video prediction, and style transfer applications.
d. Variational autoencoders (VAEs): These models encode input data into a latent space (a compressed representation) and then decode it back into output data. The latent space can be manipulated to generate new variations of the output data. They may use self-attention mechanisms to process input data, allowing them to handle long text sequences and capture complex dependencies.
e. Transformer models: These models use attention mechanisms to learn the relationships between different parts of input data (such as words or pixels) and generate output data based on these relationships. Transformer models can handle sequential data, such as text or speech, and non-sequential data, such as images or code. In an example, at least one of the models used herein is a transformer model.

In generative AI examples, the prediction/inference data that is output includes trend assessment and predictions, translations, summaries, image or video recognition and categorization, natural language processing, face recognition, user sentiment assessments, advertisement targeting and optimization, voice recognition, or media content generation, recommendation, and personalization.

The machine learning model (or generative AI) can have access to a wide variety of patient information that is stored in the database, e.g., codes. The machine learning model can process a wide variety of patient information and extract a portion of the patient information based on a set of prompts.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present (either spatially or functionally) between the first and second elements.

As noted below, the term “set” generally means a grouping of one or more elements. However, in various implementations a “set” may, in certain circumstances, be the empty set (in other words, the set has zero elements in those circumstances). As an example, a set of search results resulting from a query may, depending on the query, be the empty set. In contexts where it is not otherwise clear, the term “non-empty set” can be used to explicitly denote exclusion of the empty set—that is, a non-empty set will always have one or more elements.

A “subset” of a first set generally includes some of the elements of the first set. In various implementations, a subset of the first set is not necessarily a proper subset: in certain circumstances, the subset may be coextensive with (equal to) the first set (in other words, the subset may include the same elements as the first set). In contexts where it is not otherwise clear, the term “proper subset” can be used to explicitly denote that a subset of the first set must exclude at least one of the elements of the first set. Further, in various implementations, the term “subset” does not necessarily exclude the empty set. As an example, consider a set of candidates that was selected based on first criteria and a subset of the set of candidates that was selected based on second criteria; if no elements of the set of candidates met the second criteria, the subset may be the empty set. In contexts where it is not otherwise clear, the term “non-empty subset” can be used to explicitly denote exclusion of the empty set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” can be replaced with the term “controller” or the term “circuit.” In this application, the term “controller” can be replaced with the term “module.”

The term “module” may refer to, be part of, or include processor hardware (shared, dedicated, or group) that executes code coupled with memory hardware (shared, dedicated, or group) that stores code executed by the processor hardware. In some examples, the term “module” may refer to, be part of, or include circuitry, e.g., processor circuitry, communication circuitry, circuitry representing nodes in an artificial intelligence engine that executes instructions coupled with memory circuitry that stores instructions code executed by the circuitry and data input or output from the circuitry.

The module may include one or more interface circuit(s). In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module. The term memory hardware is a subset of the term computer-readable medium.

The apparatuses and methods described in this application may be partially or fully implemented by a special purpose computer created by configuring a general purpose computer to execute one or more particular functions embodied in computer programs. Such apparatuses and methods may be described as computerized or computer-implemented apparatuses and methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit, or buffers), electrical charge storage, magnetic storage media (such as an analog or digital magnetic tape or a hard disk drive), and optically readable storage media (such as a CD, a DVD, or a Blu-ray Disc). A non-transitory computer-readable medium can further include an automated readable format or machine-readable format. Any process, or partial steps in a process, described herein can be stored as instructions on the medium.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR.

1. A computer-implemented method for generating a prediction for a specified entity, the method comprising:
generating a base code embedding in a first vector space for the specified entity using at least one first machine learning model based on a set of codes corresponding to the specified entity from a claims datastore, wherein the at least one first machine learning model is configured to generate the base code embeddings such that semantically similar codes are closer in the first vector space;
generating an event embedding in a second vector space for the specified entity using a second machine learning model based on a set of historical events corresponding to the specified entity and the base code embedding, wherein the second machine learning model is configured to generate the event embedding such that codes commonly found in a single event are closer in the second vector space;
generating a time embedding in a third vector space for the specified entity using a third machine learning model based on times between consecutive ones of the set of historical events and the base code embedding, wherein the third machine learning model is configured to generate the time embedding such that sets of similarly spaced events are closer in the third vector space;
generating an aggregated embedding for the specified entity based on the event embedding and the time embedding; and
in response to a query designating the specified entity, generating the prediction by supplying the aggregated embedding to a fourth machine learning model.

2. The method of claim 1 wherein:
the set of codes includes more than 10,000 codes; and
the set of codes is associated with International Statistical Classification of Diseases tenth revision (ICD-10) codes.

3. The method of claim 1 wherein:
the set of codes includes more than 10,000 codes; and
the set of codes is associated with Current Procedural Terminology (CPT) codes.

4. The method of claim 1 wherein the generating the base code embedding includes:
generating tokens of the set of codes;
inputting the tokens into fifth machine learning model, a sixth machine learning model, and a seventh machine learning model;
generating a code embedding, via the fifth machine learning model, representing a first subset of the set of codes that includes whole codes;
transmitting the code embedding to an embeddings combiner;
inputting the code embedding into the sixth machine learning model;
generating a prefix embedding, via the sixth machine learning model, representing a second subset of the set of codes that includes partial codes;
transmitting the prefix embedding to the embeddings combiner;
generating a character embedding, via the seventh machine learning model, representing a third subset of the set of codes that includes character codes;
transmitting the character embedding to the embeddings combiner; and
generating the base code embedding, via the embeddings combiner, by concatenating the code embedding, the prefix embedding, and the character embedding.

5. The method of claim 1 wherein the generating the event embedding includes aggregating portions of the base code embedding associated with visits to medical provides for a plurality of patients.

6. The method of claim 1 further comprising, in response to the query, obtaining the aggregated embedding from a data store.

7. The method of claim 1 further comprising:
transforming the prediction for display on a user device; and
displaying the transformed prediction on the user device.

8. The method of claim 1 wherein the prediction includes a future cost prediction for the specified entity.

9. The method of claim 1 wherein the prediction includes a patient similarity prediction for the specified entity.

10. The method of claim 1 wherein the prediction includes a clinical insights prediction for the specified entity.

11. A computer system comprising:
memory hardware configured to store instructions; and
processor hardware configured to execute the instructions, wherein the instructions include:
generating a base code embedding in a first vector space for a specified entity using at least one first machine learning model based on a set of codes corresponding to the specified entity from a claims datastore, wherein the at least one first machine learning model is configured to generate the base code embeddings such that semantically similar codes are closer in the first vector space;
generating an event embedding in a second vector space for the specified entity using a second machine learning model based on a set of historical events corresponding to the specified entity and the base code embedding, wherein the second machine learning model is configured to generate the event embedding such that codes commonly found in a single event are closer in the second vector space;
generating a time embedding in a third vector space for the specified entity using a third machine learning model based on times between consecutive ones of the set of historical events and the base code embedding, wherein the third machine learning model is configured to generate the time embedding such that sets of similarly spaced events are closer in the third vector space;
generating an aggregated embedding for the specified entity based on the event embedding and the time embedding; and
in response to a query designating the specified entity, generating a prediction by supplying the aggregated embedding to a fourth machine learning model.

12. The computer system of claim 11 wherein:
the set of codes includes more than 10,000 codes; and
the set of codes is associated with at least one of International Statistical Classification of Diseases tenth revision (ICD-10) codes or Current Procedural Terminology (CPT) codes.

13. The computer system of claim 11 wherein the generating the base code embedding includes:
generating tokens of the set of codes;
inputting the tokens into fifth machine learning model, a sixth machine learning model, and a seventh machine learning model;
generating a code embedding, via the fifth machine learning model, representing a first subset of the set of codes that includes whole codes;
transmitting the code embedding to an embeddings combiner;
inputting the code embedding into the sixth machine learning model;
generating a prefix embedding, via the sixth machine learning model, representing a second subset of the set of codes that includes partial codes;
transmitting the prefix embedding to the embeddings combiner;
generating a character embedding, via the seventh machine learning model, representing a third subset of the set of codes that includes character codes;
transmitting the character embedding to the embeddings combiner; and
generating the base code embedding, via the embeddings combiner, by concatenating the code embedding, the prefix embedding, and the character embedding.

14. The computer system of claim 11 wherein the generating the event embedding includes aggregating portions of the base code embedding associated with visits to medical provides for a plurality of patients.

15. The computer system of claim 11 wherein the instructions further include, in response to the query, obtaining the aggregated embedding from a data store.

16. The computer system of claim 11 wherein the instructions further include:
transforming the prediction for display on a user device; and
displaying the transformed prediction on the user device.

17. The computer system of claim 11 wherein the prediction includes a future cost prediction for the specified entity.

18. The computer system of claim 11 wherein the prediction includes a patient similarity prediction for the specified entity.

19. The computer system of claim 11 wherein the prediction includes a clinical insights prediction for the specified entity.

20. A non-transitory computer-readable medium comprising processor-executable instructions that include:
generating a base code embedding in a first vector space for a specified entity using at least one first machine learning model based on a set of codes corresponding to the specified entity from a claims datastore, wherein the at least one first machine learning model is configured to generate the base code embeddings such that semantically similar codes are closer in the first vector space;
generating an event embedding in a second vector space for the specified entity using a second machine learning model based on a set of historical events corresponding to the specified entity and the base code embedding, wherein the second machine learning model is configured to generate the event embedding such that codes commonly found in a single event are closer in the second vector space;
generating a time embedding in a third vector space for the specified entity using a third machine learning model based on times between consecutive ones of the set of historical events and the base code embedding, wherein the third machine learning model is configured to generate the time embedding such that sets of similarly spaced events are closer in the third vector space;
generating an aggregated embedding for the specified entity based on the event embedding and the time embedding; and
in response to a query designating the specified entity, generating a prediction by supplying the aggregated embedding to a fourth machine learning model.
